(19) United States
(12) Patent Application Publication
(10) Pub. No.: US 2011/0144689 A1
(43) Pub. Date: Jun. 16, 2011

(54) Occlusion Device

(75) Inventors: Andrew P. Isch, West Lafayette, IN (US); Steven J. Charlebois, West Lafayette, IN (US); James D. Purdy, Lafayette, IN (US)

(73) Assignee: MED Institute, Inc., West Lafayette, IN (US)

(21) Appl. No.: 12/638,298

(22) Filed: Dec. 15, 2009

(51) Int. Cl. A61M 29/00; A61F 2/84

(52) U.S. Cl. 606/194; 623/1.23

(57) ABSTRACT

An occlusion device includes a tubular expandable body with a frame that has a plurality of interconnected members configured to expand within a body vessel and to collapse for delivery or retrieval of the device. The occlusion device further includes a hydrophilic polyurethane hydrogel layer attached to the interconnected members of the tubular expandable body. The polyurethane hydrogel layer expands upon exposure to an aqueous environment.

### BACKGROUND

The present invention generally relates to vascular occlusion devices. More specifically, the invention relates to occlusion devices having an expandable body.

A number of different devices may be used to occlude a body cavity, for example, a blood vessel. When it is desirable to quickly occlude a blood vessel, an inflatable balloon may be used. However, balloons have the disadvantage of being temporary. Another example of an occlusion device includes embolization coils. Embolization coils may be permanent and promote blood clots or tissue growth over a period of time, thereby occluding the body cavity. In conjunction with the embolization coil, a spider shaped vascular obstruction device may be used to prevent dislodgment of the embolization coils while the blood clots or the tissue grows. However, with this arrangement the blood may continue to flow past the coil and spider shaped device and through the body cavity until it finally occludes. It may take a significant period of time for sufficient clotting or tissue growth to fully occlude the body cavity. This leaves a patient open to a risk of injury from the condition which requires the body cavity to be occluded. Also, this arrangement is more complex since it requires the delivery of two or more separate devices to the vasculature.

### SUMMARY

In one form, an occlusion device includes a tubular expandable body with a frame that has a plurality of interconnected members configured to expand within the body vessel and to collapse for delivery or retrieval of the device. The occlusion device further includes a hydrophilic polyurethane hydrogel layer attached to the interconnected members of the tubular expandable body. The polyurethane hydrogel layer expands upon exposure to an aqueous environment.

The present invention also encompasses a delivery assembly for placing and retrieving the occlusion device into a body vessel. The assembly includes an outer sheath having a tubular body extending from a proximal part to a distal part and including a sheath lumen. An inner member extends from a proximal portion to a distal portion and is disposed within the sheath lumen and configured for axial movement relative to the outer sheath. The occlusion device is coaxially disposed within the sheath lumen and removably coupled to the distal portion of the inner member and is deployable through the distal part of the outer sheath by means of the relative axial movement of the inner member. The occlusion device includes any of the devices described herein.

The present invention also includes a method of constructing an occlusion device for occluding a body vessel. The method includes cutting a tubular expandable body to form a frame with interconnected members. The method further includes inserting a mandrel into one or both ends of the expandable body to form the body into a predetermined shape.

Further features and advantages of this invention will become readily apparent from the following description and from the claims.

### BRIEF DESCRIPTION OF THE DRAWINGS

FIG. 1a is a side view of a tubular expandable body that may be used to form an occlusion device in accordance with the principles of the present invention;

FIG. 1b is an end view of the tubular expandable body of FIG. 1a in accordance with the principles of the present invention;

FIG. 1c is a side view of the tubular expandable body of FIG. 1a in a collapsed state in accordance with the principles of the present invention;

FIG. 2a is a side view of an occlusion device embodying the principles of the present invention, which includes the tubular expandable body of FIGS. 1a-1b;

FIG. 2b is an end view of the occlusion device of FIG. 2a, in accordance with the principles of the present invention;

FIG. 3 is a side view of the occlusion device of FIGS. 2a and 2b partially collapsed inside of a catheter sheath in accordance with the principles of the present invention;

FIG. 4 is a cross-sectional view of the catheter sheath of FIG. 3, showing the occlusion device of FIGS. 2a, 2b, and 3, the occlusion device being collapsed inside of the catheter sheath in accordance with the principles of the present invention;

FIG. 5 is a side view of another occlusion device in accordance with the principles of the present invention;

FIG. 6 is a side view of the occlusion device of FIG. 5 with a mandrel in accordance with the principles of the present invention;

FIG. 7 is a side view of the occlusion device of FIG. 5 shown with a covering in accordance with the principles of the present invention;

FIG. 8 is a side view of yet another occlusion device in accordance with the principles of the present invention;

FIG. 9 is a side view of yet another occlusion device in accordance with the principles of the present invention;

FIG. 10 is a side view of yet another occlusion device in accordance with the principles of the present invention;

FIG. 11 is a side view of yet another occlusion device in accordance with the principles of the present invention;

FIG. 12 is a side view of yet another occlusion device in accordance with the principles of the present invention, the occlusion device being disposed inside a body vessel;

FIG. 13a is a side view of a delivery and retrieval assembly for use with the occlusion device, in accordance with the principles of the present invention;

FIG. 13b is an exploded view of the delivery and retrieval assembly of FIG. 22a, in accordance with the principles of the present invention; and

FIG. 14 is a block diagram describing a method of constructing an occlusion device, in accordance with the principles of the present invention.

### DETAILED DESCRIPTION

The terms “about” or “substantially” used herein with reference to a quantity includes variations in the recited quantity that are equivalent to the quantity recited, such as an amount that is insubstantially different from a recited quantity for an intended purpose or function.

Referring now to FIGS. 2a and 2b, a first embodiment of an occlusion device for occluding a body vessel or another body lumen, such as an aneurysm, is illustrated therein and designated at 30. As its primary components, the occlusion device 30 includes a tubular expandable body 32 with an unexpanded middle portion 46.

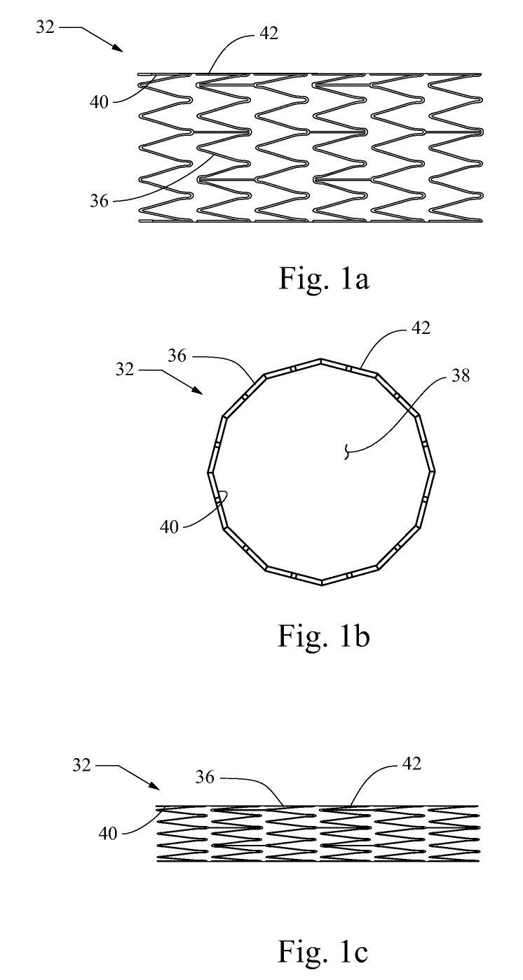

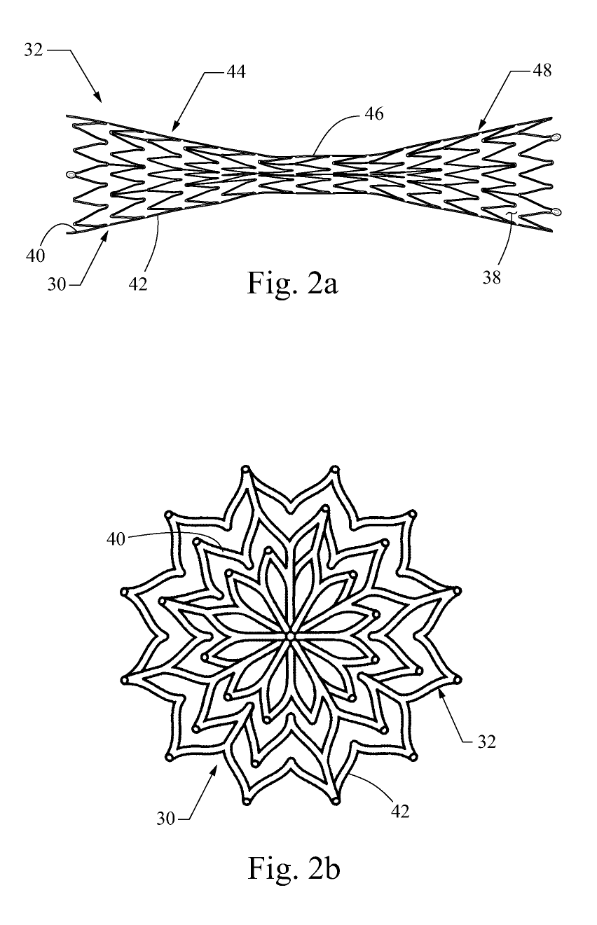

The tubular expandable body 32 of the occlusion device 30 may be frame-based, as shown in FIGS. 1a and 1b, wherein the tubular expandable body 32 comprises a frame having a plurality of members 36, such as wires, that are interconnected and configured to expand into an open configuration and are collapsible into a collapsed configuration. The members 36 of the frame define a lumen 38 therethrough. As such, the tubular expandable body 32 has an interior side 40 and an exterior side 42. Preferably, the tubular expandable body 32 is cylindrical, although other configurations may be used, without falling beyond the spirit and scope of the present invention. Although the members 36 of the tubular expandable body 32 are shown having zigzag shapes, many other configurations may be suitable, such as those disclosed in U.S. Pat. No. 4,580,568; U.S. Pat. No. 5,035,706; U.S. Pat. No. 5,507,767; and U.S. Pat. No. 6,042,606 all of which are incorporated herein by reference in their entireties. For example, the members 36 could alternatively have a sinusoidal shape or a criss-cross pattern. The tubular expandable body 32 could be formed in different ways, which also affects its configuration. For example, the tubular expandable body could be cut from a thin solid tube, such that it expands to a much larger tube having a lumen formed therethrough. In such a configuration, the tubular expandable body 32 is collapsible down to nearly the size of the original thin solid tube that it was formed from. In the alterative, the tubular expandable body could be formed from a plurality of braided members.

The tubular expandable body 32 may be made of any suitable material, for example, a superelastic material, a nickel-based superalloy, stainless steel wire, cobalt-chromium-nickel-molybdenum-iron alloy, cobalt chrome-alloy, stress relieved metal (e.g., platinum), or nickel-based superalloys, such as Inconel. The tubular expandable body 32 may preferably be formed of any appropriate material that will result in a self-expanding device 30 capable of being percutaneously inserted and deployed within a body cavity, such as shape memory material. Shape memory materials or alloys have the desirable property of becoming rigid, i.e., returning to a remembered state, when heated above a transition temperature. A shape memory alloy suitable for the present invention is nickel-titanium (Ni—Ti) available under the more commonly known name Nitinol. When this material is heated above the transition temperature, the material undergoes a phase transformation from martensite to austenite, such that the material returns to its remembered state. The transition temperature is dependent on the relative proportions of the alloying elements Ni and Ti and the optional inclusion of alloying additives. The Nitinol could be of various types, such as linear elastic Nitinol or radiopaque Nitinol.

In one embodiment, the tubular expandable body 32 is made from Nitinol with a transition temperature that is slightly below normal body temperature of humans, which is about 98.6° F. Thus, when the device 30 is deployed in a body vessel and exposed to normal body temperature, the alloy of the tubular expandable body 32 will transform to austenite, that is, the remembered state, which for one embodiment of the present invention is the expanded state when the device 30 is deployed in the body vessel. To remove the device 30, it is cooled to transform the material to martensite which is more ductile than austenite, making the tubular expandable body 32 more malleable. As such, the device 30 can be more easily collapsed and pulled into a lumen of a catheter for removal.

In another embodiment, the tubular expandable body 32 is made from Nitinol with a transition temperature that is above normal body temperature of humans, which is about 98.6° F. Thus, when the device 30 is deployed in a body vessel and exposed to normal body temperature, the tubular expandable body 32 is in the martensitic state so that the tubular expandable body 32 is sufficiently ductile to bend or form into a desired shape, which for the present embodiment is the expanded state. To remove the device 30, the device 30 is heated to transform the alloy of the tubular expandable body 32 to austenite so that it becomes rigid and returns to a remembered state, which for the device 30 is a collapsed state.

With reference to FIGS. 2a and 2b, the tubular expandable body 32 may be described as having a distal portion 44, the middle portion 46, and a proximal portion 48, with the middle portion 46 being located between the proximal and distal portions 48, 44. The proximal and distal portions 48, 44 each have open ends. The middle portion 46 has a diameter that is smaller than the diameters of the proximal and distal portions 44, 48, in the expanded state. In other words, the middle portion 46 has a diameter smaller than the diameters of each of the open ends. In this embodiment, the open ends, or the proximal and distal portions 48, 44, have diameters that are about equal.

The lumen 38 is not necessarily completely closed (although it could be) in the middle portion 46, but in this embodiment the lumen 38 may be collapsed to close a majority of the through-channel of the lumen 38, so that occlusion of the body vessel may occur. The occlusion device 30 may be described as having an hour glass shape, or a bow tie shape, such that the proximal and distal portions 44, 48 are larger than the middle portion 46. Furthermore, identification eyelets with radiopaque qualities could be located on the tubular expandable body 32.

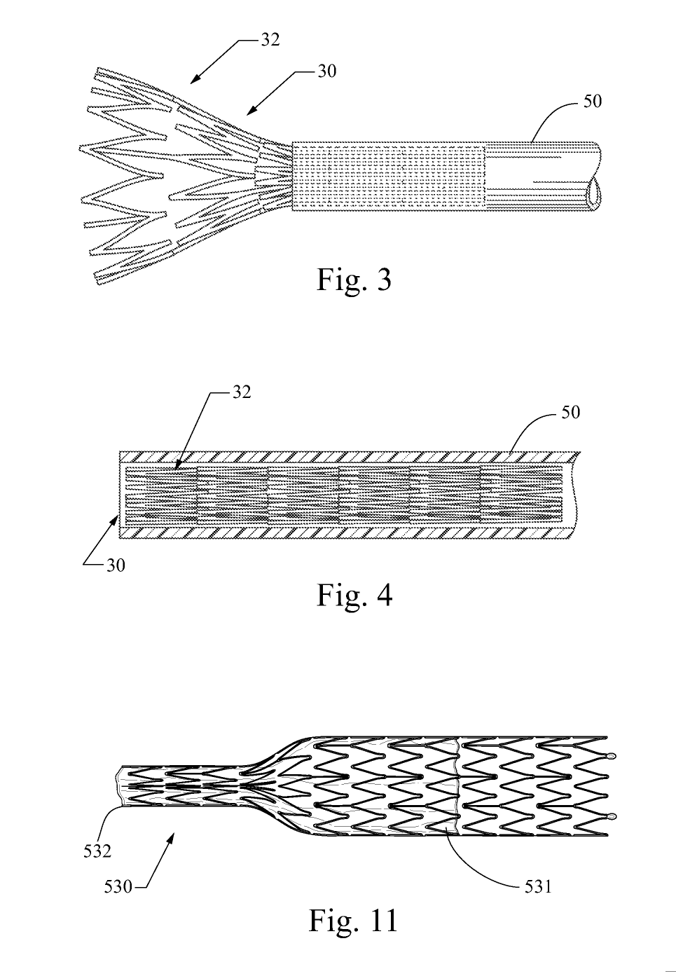

With reference to FIGS. 2a, 2b, 3 and 4, the occlusion device 30 is configured to move between an expanded state for occlusion within a body vessel and a collapsed state for delivery or retrieval of the device 30. The device 30 is configured to open radially to define the expanded state and to collapse along a central longitudinal axis, which extends through the lumen 38, to define the collapsed state. In FIGS. 2a and 2b, the occlusion device 30 is shown in the expanded state. In FIG. 3, the device 30 is partially located within a sheath 50, wherein a portion of the device 30 is collapsed and a portion of the device 30 is expanded. In FIG. 4, the device 30 is collapsed within the sheath 50 in the collapsed state. Even in the expanded state of the device 30, the middle portion 46 of the tubular expandable body 32 is collapsed.

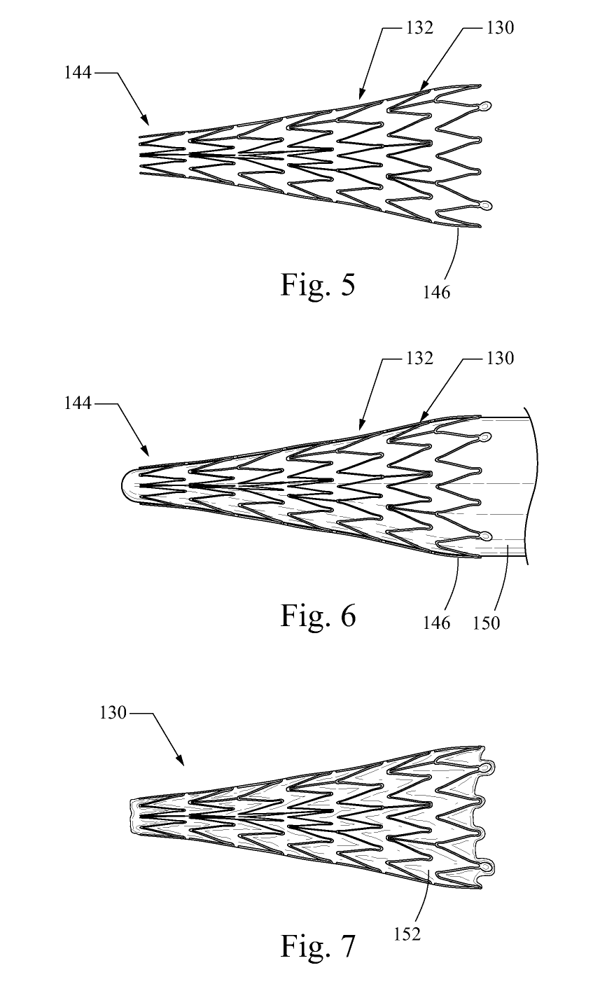

With reference to FIG. 5, another occlusion device 130 is illustrated. The occlusion device 130 has a tubular expandable body 132, which is collapsed or unexpanded at one end 144 and is opened or expanded at the other end 146. Thus, the occlusion device 130 has a conical shape. In all other respects, the occlusion device 130 may be similar to those hereinbefore or hereinafter described.

Turning to FIG. 6, the occlusion device 130 may be formed with the use of a conical-shaped mandrel 150. In particular, the mandrel 150 is inserted into one end of the tubular expandable body 32 while in its collapsed state. As the mandrel 150 moves toward the other end of the tubular expandable body 32, the tubular expandable body 32 takes the shape of the conical-shaped portion of the mandrel 150 and then heat setting the body 32 to form the occlusion device 130. A similar process may be employed to form the occlusion device 30 shown in FIGS. 2a and 2b by inserting the mandrel 150 in both ends of the body 32 towards the middle of the body 32 and then heat setting the body 32 to form the occlusion device 30.

To enhance embolization, the aforementioned occlusion devices 30 and 130, as well as the occlusion devices discussed bellow, can have a plurality of occluding materials interwoven between members 36 of the tubular expandable body 32. The occluding materials may be threads or any other suitable occluding material. The threads or occluding material may include one or more of the following: an extracelluar matrix (ECM), such as small intestinal submucosa (SIS), synthetic polyester, such as DACRON™, nylon, rayon, polyester, polytetrafluoroethylene, polyurethane, and bioremodelable material, which could be laminated, if desired. The occluding material may itself be laminated, or it could be laminated to the tubular expandable body 32.

As known, ECM is a complex structural entity surrounding and supporting cells found within tissues. More specifically, ECM includes structural proteins (for example, collagen and elastin), specialized protein (for example, fibrillin, fibronectin, and laminin), and proteoglycans, a protein core to which are attached long chains of repeating disaccharide units termed glycosaminoglycans.

In one particular embodiment, the extracellular matrix is comprised of small intestinal submucosa (SIS). As known, SIS is a resorbable, acellular, naturally occurring tissue matrix composed of extracellular matrix (ECM) proteins and various growth factors. SIS is derived from the porcine jejunum and functions as a remodeling bioscaffold for tissue repair. SIS has characteristics of an ideal tissue engineered biomaterial and can act as a bioscaffold for remodeling of many body tissues including skin, body wall, musculoskeletal structure, urinary bladder, and also supports new blood vessel growth. SIS may be used to induce site-specific remodeling of both organs and tissues depending on the site of implantation. In practice, host cells are stimulated to proliferate and differentiate into site-specific connective tissue structures, which have been shown to completely replace the SIS material in time.

SIS may attached to the occlusion devices to assist with occluding a body vessel, adhere to the walls of the body vessel in which the device is deployed, and promote body tissue growth within the body vessel. SIS has a natural adherence or wetability to body fluids and connective cells comprising the connective tissue of the walls of a body vessel. If the occlusion device is intended to permanently occlude the body vessel, the device is positioned such that the host cells of the wall will adhere to the SIS and subsequently differentiate, growing into the SIS and eventually occluding the body vessel with the tissue of the walls to which the device was originally adhered. This feature enhances permanent occlusion of the body vessel. In another particular embodiment, the SIS may be used to temporarily adhere the occlusion device to the walls of the body vessel. If the occlusion device is only deployed within the body vessel temporarily, host cells of the walls may adhere to the device, but will not differentiate, allowing for later retrieval of the device from the body vessel. The occluding material may be attached to the body 32 in any manner as described in U.S. application Ser. No. 12/034,719, filed Feb. 21, 2008, the entire contents of which is incorporated herein by reference.

With reference to FIG. 7 the occlusion device 130 is shown with a covering 152 that includes any suitable material configured to prevent blood, emboli and other fluids from passing, and thereby assist in occluding the body vessel. The covering 152 can be employed with the occlusion device 30 as well as any of the other occlusion devices described below.

In one embodiment, the covering 152 may be made of nylon, rayon, polyester, biocompatible polyurethanes, polytetrafluoroethylene (known as PTFE or under the trade name Teflon™), and mixtures thereof without falling beyond the scope or spirit of the present invention. In one example, the material may be made of one material and coated with another, such as the biocompatible polyurethane. In another example, the occluding barrier may be made of connective tissue material including, for example, extracellular matrix (ECM), which is described above.

One example of the biocompatible polyurethane is sold under the trade name THORALON (THORATEC, Pleasanton, Calif.). Descriptions of suitable biocompatible polyureaurethanes are described in U.S. Pat. Application Publication No. 2002/0065552 A1 and U.S. Pat. No. 4,675,361, both of which are herein incorporated by reference. Briefly, these publications describe a polyurethane base polymer (referred to as BPS-215) blended with a siloxane containing surface modifying additive (referred to as SMA-300). Base polymers containing urea linkages can also be used. The concentration of the surface modifying additive may be in the range of 0.5% to 5% by weight of the base polymer.

The SMA-300 component (THORATEC) is a polyurethane comprising polydimethylsiloxane as a soft segment and the reaction product of diphenylmethane diisocyanate (MDI) and 1,4-butanediol as a hard segment. A process for synthesizing SMA-300 is described, for example, in U.S. Pat. Nos. 4,861,830 and 4,675,361, which are incorporated herein by reference.

The BPS-215 component (THORATEC) is a segmented polyetherurethane urea containing a soft segment and a hard segment. The soft segment is made of polytetramethylene oxide (PTMO), and the hard segment is made from the reaction of 4,4′-diphenylmethane diisocyanate (MDI) and ethylene diamine (ED).

THORALON can be manipulated to provide either porous or non-porous THORALON. The present invention envisions the use of non-porous THORALON. Non-porous THORALON can be formed by mixing the polyetherurethane urea (BPS-215) and the surface modifying additive (SMA-300) in a solvent, such as dimethyl formamide (DMF), tetrahydrofuran (THF), dimethyacetamide (DMAC), dimethyl sulfoxide (DMSO). The composition can contain from about 5 wt % to about 40 wt % polymer, and different levels of polymer within the range can be used to fine tune the viscosity needed for a given process. The composition can contain less than 5 wt % polymer for some spray application embodiments. The entire composition can be cast as a sheet, or coated onto an article such as a mandrel or a mold. In one example, the composition can be dried to remove the solvent.

THORALON has been used in certain vascular applications and is characterized by thromboresistance, high tensile strength, low water absorption, low critical surface tension, and good flex life. THORALON is believed to be biostable and to be useful in vivo in long term blood contacting applications requiring biostability and leak resistance. Because of its flexibility, THORALON is useful in larger vessels, such as the abdominal aorta, where elasticity and compliance is beneficial.

A variety of other biocompatible polyurethanes/polycarbamates and urea linkages (hereinafter “—C(0)N or CON type polymers”) may also be employed. These include CON type polymers that preferably include a soft segment and a hard segment. The segments can be combined as copolymers or as blends. For example, CON type polymers with soft segments such as PTMO, polyethylene oxide, polypropylene oxide, polycarbonate, polyolefin, polysiloxane (i.e. polydimethylsiloxane), and other polyether soft segments made from higher homologous series of diols may be used. Mixtures of any of the soft segments may also be used. The soft segments also may have either alcohol end groups or amine end groups. The molecular weight of the soft segments may vary from about 500 to about 5,000 g/mole.

Preferably, the hard segment is formed from a diisocyanate and diamine. The diisocyanate may be represented by the formula OCN—R—NCO, where —R— may be aliphatic, aromatic, cycloaliphatic or a mixture of aliphatic and aromatic moieties. Examples of diisocyanates include MDI, tetramethylene diisocyanate, hexamethylene diisocyanate, trimethyhexamethylene diisocyanate, tetramethylxylylene diisocyanate, 4,4′-dicyclohexylmethane diisocyanate, dimer acid diisocyanate, isophorone diisocyanate, metaxylene diisocyanate, diethylbenzene diisocyanate, decamethylene 1,10 diisocyanate, cyclohexylene 1,2-diisocyanate, 2,4-toluene diisocyanate, 2,6-toluene diisocyanate, xylene diisocyanate, m-phenylene diisocyanate, hexahydrotolylene diisocyanate (and isomers), naphthylene-1,5-diisocyanate, 1-methoxyphenyl 2,4-diisocyanate, 4,4′-biphenylene diisocyanate, 3,3′-dimethoxy-4,4′-biphenyl diisocyanate and mixtures thereof.

The diamine used as a component of the hard segment includes aliphatic amines, aromatic amines and amines containing both aliphatic and aromatic moieties. For example, diamines include ethylene diamine, propane diamines, butanediamines, hexanediamines, pentane diamines, heptane diamines, octane diamines, m-xylylene diamine, 1,4-cyclohexane diamine, 2-methypentamethylene diamine, 4,4′-methylene dianiline, and mixtures thereof. The amines may also contain oxygen and/or halogen atoms in their structures.

Other applicable biocompatible polyurethanes include those using a polyol as a component of the hard segment. Polyols may be aliphatic, aromatic, cycloaliphatic or may contain a mixture of aliphatic and aromatic moieties. For example, the polyol may be ethylene glycol, diethylene glycol, triethylene glycol, 1,4-butanediol, 1,6-hexanediol, 1,8-octanediol, propylene glycols, 2,3-butylene glycol, dipropylene glycol, dibutylene glycol, glycerol, or mixtures thereof.

Biocompatible CON type polymers modified with cationic, anionic and aliphatic side chains may also be used. See, for example, U.S. Pat. No. 5,017,664. Other biocompatible CON type polymers include: segmented polyurethanes, such as BIOSPAN; polycarbonate urethanes, such as BIONATE; and polyetherurethanes, such as ELASTHANE; (all available from POLYMER TECHNOLOGY GROUP, Berkeley, Calif.).

Other biocompatible CON type polymers can include polyurethanes having siloxane segments, also referred to as a siloxane-polyurethane. Examples of polyurethanes containing siloxane segments include polyether siloxane-polyurethanes, polycarbonate siloxane-polyurethanes, and siloxane-polyurethane ureas. Specifically, examples of siloxane-polyurethane include polymers such as ELAST-EON 2 and ELAST-EON 3 (AORTECH BIOMATERIALS, Victoria, Australia); polytetramethyleneoxide (PTMO) and polydimethylsiloxane (PDMS) polyether-based aromatic siloxane-polyurethanes such as PURSIL-10, -20, and -40 TSPU; PTMO and PDMS polyether-based aliphatic siloxane-polyurethanes such as PURSIL AL-5 and AL-10 TSPU; aliphatic, hydroxy-terminated polycarbonate and PDMS polycarbonate-based siloxane-polyurethanes such as CARBOSIL-10, -20, and -40 TSPU (all available from POLYMER TECHNOLOGY GROUP). The PURSIL, PURSIL-AL, and CARBOSIL polymers are thermoplastic elastomer urethane copolymers containing siloxane in the soft segment, and the percent siloxane in the copolymer is referred to in the grade name. For example, PURSIL-10 contains 10% siloxane. These polymers are synthesized through a multi-step bulk synthesis in which PDMS is incorporated into the polymer soft segment with PTMO (PURSIL) or an aliphatic hydroxy-terminated polycarbonate (CARBOSIL). The hard segment consists of the reaction product of an aromatic diisocyanate, MDI, with a low molecular weight glycol chain extender. In the case of PURSIL-AL the hard segment is synthesized from an aliphatic diisocyanate. The polymer chains are then terminated with a siloxane or other surface modifying end group. Siloxane-polyurethanes typically have a relatively low glass transition temperature, which provides for polymeric materials having increased flexibility relative to many conventional materials. In addition, the siloxane-polyurethane can exhibit high hydrolytic and oxidative stability, including improved resistance to environmental stress cracking. Examples of siloxane-polyurethanes are disclosed in U.S. Pat. Application Publication No. 2002/0187288 A1, which is incorporated herein by reference.

In addition, any of these biocompatible CON type polymers may be end-capped with surface active end groups, such as, for example, polydimethylsiloxane, fluoropolymers, polyolefin, polyethylene oxide, or other suitable groups. See, for example the surface active end groups disclosed in U.S. Pat. No. 5,589,563, which is incorporated herein by reference. As noted above, the occluding barrier may also be made of connective tissue material including, for example, an ECM such as SIS.

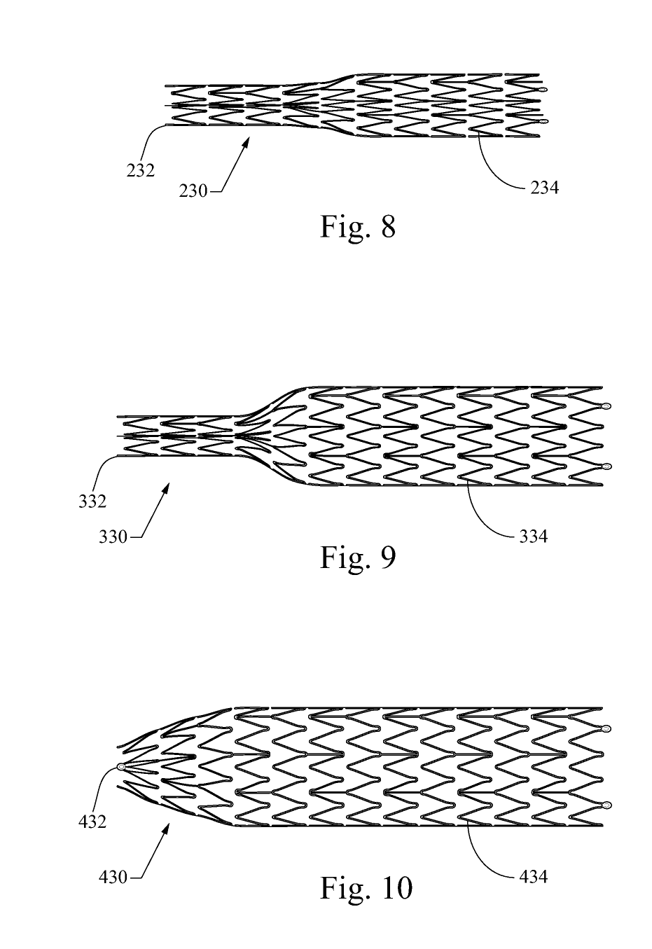

Shown in FIGS. 8 through 10 are various other configurations of occlusion devices 230, 330, and 430 that can be formed from the expandable tubular body 32 in accordance with the invention. For example, a suitably shaped mandrel may be employed to form the respective occlusion devices 230, 330, and 430 in a manner similar to that described with reference to FIG. 6.

The occlusion device 230 (FIG. 8) has an elongated tip portion 232 and a generally cylindrical body portion 234. The occlusion device 330 (FIG. 9) also has an elongated tip portion 332 and a generally cylindrical body potion 334. Although similar in shape to the occlusion device 230, the body portion 334 of the occlusion device 330 has a larger diameter and is longer than the body portion 234 of the occlusion device 230. The occlusion device 430 has a generally cylindrical body 434, but its tip portion 432 is has more of a conical shape rather than the elongated tips of the occlusion devices 230 and 330. The various shapes of the occlusion devices are formed depending on the application of the particular device.

Any of the occlusion devices described herein may have a structure with a polyurethane hydrogel layer attached to the tubular body. For example, an occlusion device 530 (FIG. 11) includes a body portion 531 embedded with a layer of polyurethane hydrogel 532. Examples of polyurethane hydrogel include HydroThane™ and HydroMed™, both of which are available from AdvanSource Biomaterials, Wilimington, Mass.

The use of a hydrophilic polyurethane hydrogel speeds up the occlusion time by further restricting the vessel pathway. Hydrophilic polyurethanes can be applied to the occlusion devices to enhance their functionality. For example, if this polymer layer is a polyurethane hydrogel, the polymer layer will enlarge or swell when exposed to an aqueous environment, thereby, allowing the struts of the occlusion device to bend more freely without kinking.

The polyurethane hydrogel layer represents a 3-dimensional network of cross-linked hydrophilic macromolecules that can swell and absorb about 20 to 90 percent by weight of water. The hydrogel layer may be applied by coating, adhesive bonding, lamination, extrusion, or molding. The application method used is selected to provide a layer of the hydrogel having a substantially uniform thickness.

The occlusion device may include an expandable structure embedded within a polymeric matrix or a solid polymeric matrix. In both cases, the polyurethane hydrogel may be applied as a strip or band disposed around the outer surface of the occlusion device. After the hydrogel is disposed around the occlusion device, it may be dried by any method known in the art, including but not limited to conduction drying, convection drying, hot air impingement, steam treatment, infrared irradiation, ultraviolet irradiation, and microwave irradiation. Preferably, the hydrogel coating is dried by the application of thermal energy.

Upon exposure to an aqueous environment, i.e., bodily fluid, the hydrogel coating will absorb water and swell to a diameter that is larger than the diameter of the elongated body, such as the body portion 531 of the occlusion device 530. The increase in diameter the polyurethane hydrogel layer upon exposure to an aqueous environment can be on the order of about 10% to 30%.

The hydrophilic polyurethane hydrogel can be applied to the occlusion device as part of their manufacturing operation or applied to devices that have already been manufactured. In this latter case, the hydrogel may be applied to the surface of the device. The polyurethane hydrogel may also be used as the base polymer from which the device is extruded.

Since a polyurethane hydrogel can be extruded at a lower temperature than a conventional polyurethane material, the polyurethane hydrogel can be loaded with a therapeutic agent prior to extrusion, because the therapeutic agent is capable of avoiding degradation during the extrusion process.

The occlusion device may include an expandable mesh upon which the polyurethane hydrogel is applied and disposed within the interstices of the mesh. This expandable mesh can be made from braided filaments, a coiled spring, or any other expandable arrangement that may be collapsed and when released expands radially. The expandable mesh may be made from any suitable material, such as stainless steel, tantalum, gold, titanium, and Nitinol. The expandable mesh should be designed such that it will have exhibit approximately the same or similar expansion ratio as the polyurethane hydrogel that is utilized in order to allow for full expansion with minimum resistance.

The polyurethane hydrogel may optionally include a therapeutic agent for treatment, such as, for example chemotherapy, of a tumor, at or near the site where the occlusion device is implanted. The therapeutic agent may be an anti-platelet, anti-coagulant, anti-betabolite, anti-aniogenic, anti-thrombogenic, or anti-proliferative drug.

The exterior of the occlusion device may optionally include one or more radiopaque or echogenic features, such as a marker used to detect positioning of the device via a suitable imaging technique. The radiopaque or echogenic feature may be applied by any fabrication method or absorbed into or sprayed onto the surface of the device. Common radiopaque materials include barium sulfate and zirconium dioxide, as well as various elements, such as cadmium, tungsten, gold, tantalum, bismuth, platinum, iridium, and rhodium.

Other features of devices using polyurethane hydrogel may be found in U.S. Provisional Patent Application No. 61/223,418, filed Jul. 7, 2009, the entire contents of which are incorporated herein by reference.

In any occlusion device described herein, the tubular expandable body may include a plurality of portions having varying amounts of stiffness. In order to have varying amounts of stiffness, a tubular expandable body could have members which have different thicknesses or a thickness that varies along the length of a member. In addition, or in the alternative, part of the tubular expandable body, as used for the invention herein, could be annealed to make parts of the body structure softer. In another variation, the tubular expandable body could have a varying wire design, or different kinds of cuts, to provide areas that are softer than others.

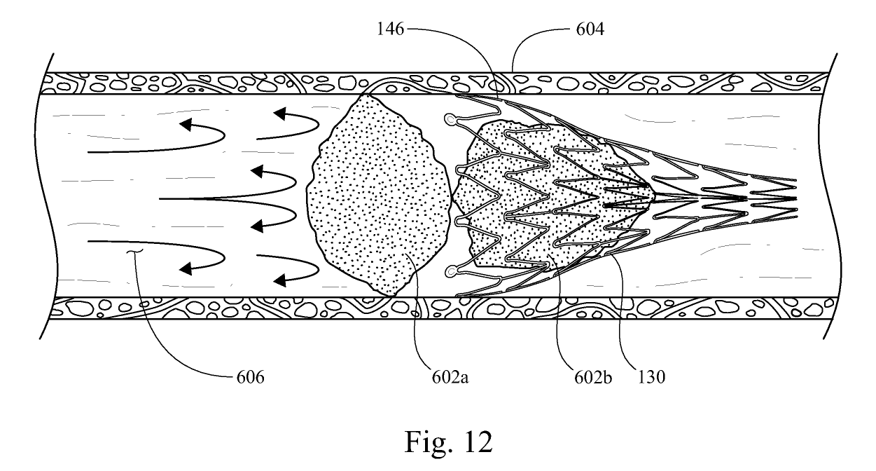

Referring to FIG. 12, another implementation of the occlusion device 130 in accordance with the invention is shown deployed in a blood vessel 604. Positioned within and/or toward the end 146 of the device 130 are one or more plugs of SHISH material 602a and 602b, which is a crosslinked and stabilized, foam-like SIS material (described previously) that swells when hydrated and is based on the same extracellular matrix technology as SIS.

Before deployment in the blood vessel 604, the SHISH material 602a and 602b is an unexpanded state. As the SHISH material begins to hydrate, it expands and becomes softer to conform to the inside of the body of the occlusion device 130 to further occlude blood flow 606. Clotting then proceeds because of the blocked blood flow. Although the occlusion device 130 is shown in FIG. 12 for this particular implementation, any of the other aforementioned occlusion devices may be used in combination with the SHISH plugs.

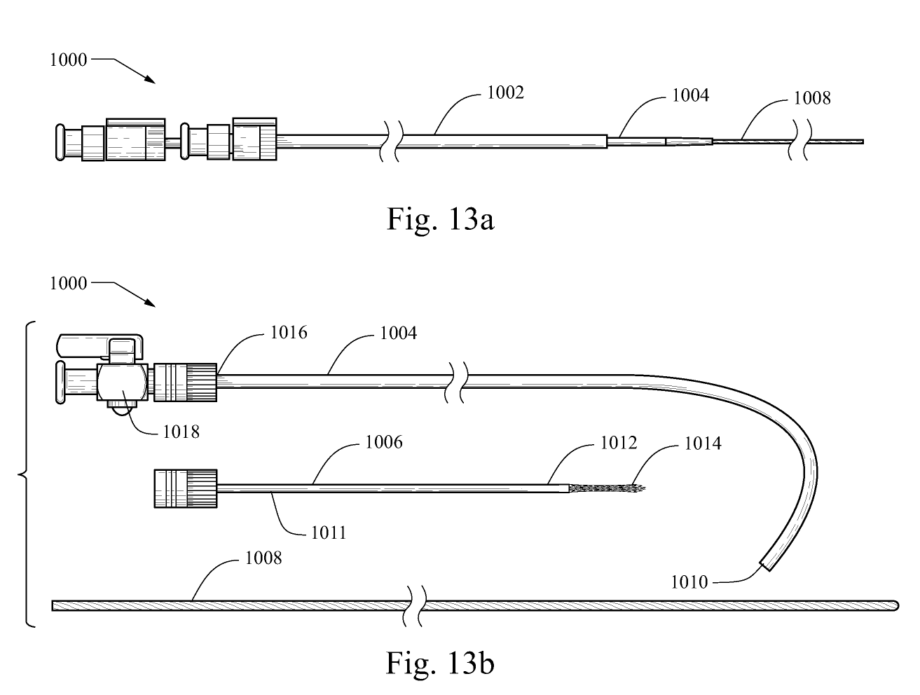

FIGS. 13a and 13b depict a delivery assembly 1000 for introducing and retrieving the occlusion device for occluding a body vessel in accordance with another embodiment of the present invention. As shown, the delivery assembly 1000 includes a polytetrafluoroethylene (PTFE) introducer sheath 1002 for percutaneously introducing an outer sheath 1004 (equivalent to the sheath 50 described above) into a body vessel. Of course, any other suitable material for the introducer sheath 1002 may be used without falling beyond the scope or spirit of the present invention. The introducer sheath 1002 may have any suitable size, for example, between about three-french to eight-french. The introducer sheath 1002 serves to allow the outer sheath 1004 and an inner member or catheter 1006 to be percutaneously inserted to a desired location in the body vessel. The inner member may also include, for example, a stylet. The introducer sheath 1002 receives the outer sheath 1004 and provides stability to the outer sheath 1004 at a desired location of the body vessel. For example, the introducer sheath 1002 is held stationary within a common visceral artery, and adds stability to the outer sheath 1004, as the outer sheath 1004 is advanced through the introducer sheath 1002 to an occlusion area in the vasculature. The outer sheath 1004 has a body extending from a proximal end 1016 to a distal end 1010, the body being tubular and including a sheath lumen extending therethrough.

As shown, the assembly 1000 may also include a wire guide 1008 configured to be percutaneously inserted within the vasculature to guide the outer sheath 1004 to the occlusion area. The wire guide 1008 provides the outer sheath 1004 with a path to follow as it is advanced within the body vessel. The size of the wire guide 1008 is based on the inside diameter of the outer sheath 1004 and the diameter of the target body vessel.

When the distal end 1010 of the outer sheath 1004 is at the desired location in the body vessel, the wire guide 1008 is removed and the occlusion device 1014, having a proximal segment contacting a distal portion 1012 of the inner catheter 1006, is inserted into the outer sheath 1004. The inner catheter 1006 is advanced through the outer sheath 1004 for deployment of the occlusion device 1014 through the distal end 1010 to occlude the body vessel during treatment of, for example, an aneurism, or to otherwise occlude a body vessel. The catheter 1006 extends from a proximal portion 1011 to a distal portion 1012 and is configured for axial movement relative to the outer sheath 1004. In this example, the distal portion 1012 is shown adjacent to an occlusion device 1014 (similar to any of the occlusion devices described above). Thus, before deployment, the occlusion device 1014 is coaxially disposed within the lumen of the outer sheath 1004 and removably coupled to the distal portion 1012 of the catheter 1006, or in the alternative, the occlusion device 1014 is merely pushed by, but not coupled to, the distal portion 1012 of the catheter 1006.

The outer sheath 1004 further has a proximal end 1016 and a hub 1018 to receive the inner catheter 1006 and occlusion device 1014 to be advanced therethrough. The size of the outer sheath 1004 is based on the size of the body vessel in which it percutaneously inserts, and the size of the occlusion device 1014.

In this embodiment, the occlusion device 1014 and inner catheter 1006 are coaxially advanced through the outer sheath 1004, following removal of the wire guide 1008, in order to position the occlusion device 1014 to occlude the body vessel. The occlusion device 1014 is guided through the outer sheath 1004 by the inner catheter 1006, preferably from the hub 1018, and exits from the distal end 1010 of the outer sheath 1004 at a location within the vasculature where occlusion is desired. Thus, the occlusion device 1014 is deployable through the distal end 1010 of the outer sheath 1004 by means of axial relative movement of the catheter 1006. In order to more easily deploy the occlusion device 1014 into the body vessel, the occlusion device 1014 may have a slippery coating, such as Silicone, slipcoating, hydrogel, or hydrophilic coating.

Likewise, this embodiment may also retrieve the occlusion device 1014 by positioning the distal end 1010 of the outer sheath 1004 adjacent the deployed device 1014 in the vasculature. The inner catheter 1006 is advanced through the outer sheath 1004 until the distal portion 1012 protrudes from the distal end 1010 of the outer sheath 1004. The distal portion 1012 is coupled to a proximal end of the occlusion device 1014, after which the inner catheter 1006 is retracted proximally, drawing the occlusion device 1014 into the outer sheath 1004.

In a particular arrangement, SHISH plugs (FIG. 12) are deployed along with the occlusion device. In such an arrangement, the SHISH plugs are loaded behind the occlusion device 1014, that is, between the distal portion 1012 and the proximal end of the occlusion device 1014. During deployment, the SHISH plugs are positioned into the expanded occlusion device 1014 by natural blood flow or by using the catheter tip to nudge the SHISH plug(s) into position.

It is understood that the assembly described above is merely one example of an assembly that may be used to deploy the occlusion device in a body vessel. Of course, other apparatus, assemblies and systems may be used to deploy any embodiment of the occlusion device without falling beyond the scope or spirit of the present invention.

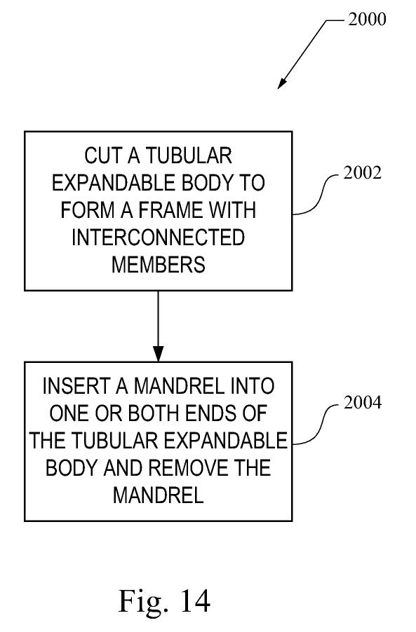

Turning to FIG. 14, in accordance with the invention a method 2000 of constructing an occlusion device for occluding a body vessel is shown. The method 2000 includes a step 2002 of cutting, for example, with a laser, a tubular expandable body to form a frame with interconnected members, so that the tubular expandable body defines a lumen along a longitudinal axis through a center of the tubular expandable body, and a step 2004 of inserting a mandrel into one or both ends of the tubular expandable body to form the body into a desired or predetermined shape according to the application of the occlusion device. The method 2000 can further include adding occluding material, of the various types described above, for example the hydrophilic polyurethane hydrogel or the SHISH material. In addition, the method 2000 can further include heat treating the tubular expandable body. In the alternative, the method 2000 can further include cold working the tubular expandable body.

As a person skilled in the art will readily appreciate, the above description is meant as an illustration of implementation of the principles this invention. This description is not intended to limit the scope or application of this invention in that the invention is susceptible to modification, variation and change, without departing from the spirit of this invention, as defined in the following claims.

What is claimed is:

1. An occlusion device for occluding a body vessel, the occlusion device comprising:
a tubular expandable body with an interior side and an exterior side, the tubular expandable body having a frame with a plurality of interconnected members configured to expand within the body vessel and to collapse for delivery or retrieval of the device; and
a hydrophilic polyurethane hydrogel layer attached to the interconnected members of the tubular expandable body, the polyurethane hydrogel layer expanding upon exposure to an aqueous environment.

2. The occlusion device of claim 1, wherein the hydrogel layer expands to an outer diameter that is greater than the outer diameter of the tubular expandable body to exert a sealing force against the interior wall of the body vessel.

3. The occlusion device of claim 2, wherein the increase in diameter of the hydrogel layer is between about 10% to 30%.

4. The occlusion device of claim 1, wherein the hydrogel layer is embedded with the tubular expandable body.

5. The occlusion device of claim 1, wherein the hydrogel layer is disposed about the interior side of the tubular expandable body.

6. The occlusion device of claim 1, wherein the hydrogel layer is disposed about the exterior side of the tubular expandable body.

7. The occlusion device of claim 1, wherein the hydrogel layer includes a therapeutic agent for chemotherapy treatment of a tumor at or near the site of the implantation of the device.

8. The occlusion device of claim 1, wherein at least a portion of the tubular expandable body is heat treated.

9. The occlusion device of claim 1, wherein the expandable tubular body has at least one generally conical portion in the expanded state.

10. The occlusion device of claim 9, wherein the device is bidirectional and the expandable tubular body has two generally conical portions in the expanded state.

11. The occlusion device of claim 1, wherein the expandable tubular body has a generally cylindrical body portion.

12. The occlusion device of claim 11, wherein the expandable tubular body has an elongated tip portion.

13. An occlusion device for occluding a body vessel, the occlusion device comprising:
a tubular expandable body with an interior side and an exterior side, the tubular expandable body having a frame with a plurality of interconnected members configured to expand within the body vessel and to collapse for delivery or retrieval of the device; and
at least one plug of SHISH disposed within the interior side of the tubular expandable body, the at least one plug of SHISH expanding upon exposure to an aqueous environment.

14. The occlusion device of claim 13, as the SHISH plug hydrates, it becomes softer and conforms to the interior side of the tubular expandable body to occlude the flow of fluid in the body vessel.

15. The occlusion device of claim 13, wherein the occlusion device includes multiple plugs of SHISH.

16. A delivery assembly for placing and retrieving an occlusion device for occluding a body vessel, the assembly comprising:
an outer sheath having a body extending from a proximal part to a distal part, the body being tubular and forming a sheath lumen extending therethrough;
an inner member extending from a proximal portion to a distal portion, the inner member being disposed within the sheath lumen and configured for axial movement relative to the outer sheath;
the occlusion device being coaxially disposed within the sheath lumen and removably coupled to the distal portion of the inner member and deployable through the distal part of the outer sheath by means of the relative axial movement of the inner member, the occlusion device comprising:
a tubular expandable body with an interior side and an exterior side, the tubular expandable body having a frame with a plurality of interconnected members configured to expand within the body vessel and to collapse for delivery or retrieval of the device; and
a hydrophilic polyurethane hydrogel layer is attached to the interconnected members of the tubular expandable body, the polyurethane hydrogel layer expanding upon exposure to an aqueous environment.

17. A method of forming an occlusion device comprising:
cutting a tubular expandable body to form a frame with interconnected members; and
inserting a mandrel into at least one end of the body to form the body into a predetermined shape.

18. The method of claim 17 further comprising heat treating the tubular expandable body.

19. The method of claim 17, wherein cutting the tubular expandable body includes cutting with a laser.

20. The method of claim 17 further comprising attaching a hydrophilic polyurethane hydrogel layer to the interconnected members of the tubular expandable body.
